(19) United States
(12) Patent Application Publication
(10) Pub. No.: US 2018/0235454 A1
(43) Pub. Date: Aug. 23, 2018

(54) SYSTEM AND METHOD FOR ENDOSCOPE HEATING

(71) Applicant: Medeon Biodesign, Inc., Taipei (TW)

(72) Inventors: Kuen-Shian Tsai, Taipei (TW); Hung-Wen Wei, Taipei (TW); Chih-Wei Hsu, Taipei (TW); Shuling Cheng, Taipei (TW)

(21) Appl. No.: 15/762,011

(22) PCT Filed: Sep. 21, 2016
(86) PCT No.: PCT/US16/52882

§ 371 Date: Mar. 21, 2018

Related U.S. Application Data
(60) Provisional application No. 62/221,860, filed on Sep. 22, 2015.

(51) Int. Cl. A61B 1/12

(52) U.S. Cl. CPC A61B 1/127; A61B 1/128

(57) ABSTRACT

Systems are methods are provided for heating an endoscope to defog or avoid from fogging of lens. The endoscope heater may have an elongated body that attaches to the shaft of an endoscope with a controller to operate a heating element disposed on the body. The controller may maintain the heating element at a predetermined temperature. In use, the controller may be operated to energize the heating element and warm the endoscope above ambient temperature prior to introduction into the patient's body.

### RELATED APPLICATIONS

This application claims the benefit of U.S. Provisional Patent Application No. 62/221,860 for “ENDOSCOPE HEATER AND METHOD OF HEATING,” filed Sep. 22, 2015, the contents of which are incorporated by reference in its entirety.

### FIELD OF THE PRESENT DISCLOSURE

The present disclosure relates generally to devices and methods for use in conjunction with an endoscope for performing laparoscopic surgical procedures. In particular, techniques for heating the distal end of an endoscope are disclosed to avoid from fogging of or defog optical elements of the endoscope during surgery.

### BACKGROUND

Laparoscopic surgery is a minimally invasive alternative to conventional “open” surgeries and provides the benefits of reducing post-operative pain, decreasing hospital stays and periods of disability, and lowering costs for both hospitals and patients. Generally, these procedures utilize an endoscope to view interior areas in the body that would not otherwise be visible, allowing access to desired locations within a patient's body. Over 7.5 million laparoscopic surgeries are performed worldwide each year in a variety of interventional and diagnostic procedures, including cholecystectomy, appendectomy, bariatric surgeries, gynecological surgeries, and urological surgeries for example.

During the laparoscopic surgery, the abdomen of the patient is typically inflated with a gas, e.g. carbon dioxide, to provide sufficient operation space to ensure adequate visualization of the structures and manipulation of instruments. A typical laparoscope features an elongated shaft with an objective lens located at the distal end. During the surgical procedure, the distal portion of the laparoscope is inserted into a patient's body while the proximal portion of the laparoscope remains outside the body to allow manipulation by the surgeon. However, the inner environment of patient's abdomen is usually warm and humid relative to the ambient environment. Thus, when the laparoscope is inserted into a patient, fogging of the objective lens may occur due to temperature and/or humidity differences between the ambient environment and the patient's body. In addition, it is sometimes necessary to supply water or air to the body cavity to remove foreign matter. The addition of water or air to the body cavity may lower the temperature of the objective lens, creating conditions that may contribute to lens fogging.

Conventional approaches to laparoscope fogging may require the surgeon to remove the instrument from the body cavity and warm the distal end to defog the lens. For example, hot water may be used to warm the lens. The surgeon may then clean the lens by wiping it with a cloth. As will be appreciated, these operations increase the amount of time required to complete the procedure, particularly if the defogging operation needs to be repeated. Further, withdrawing and reinserting the laparoscope may elevate the risk of introducing infectious materials into the patient's body or cause additional trauma.

Correspondingly, what has been needed, therefore, is an endosope heater and method of heating endoscope for anti-fogging on the lens. The endoscope heater is an accessary element for endoscope which is detachable, easy-use, and no need to be removed from patient's body to defog during surgery.

### SUMMARY

This disclosure includes an endoscope heater, which may have an elongated body configured to be attached to an endoscope shaft, wherein a surface of the body conforms to an outer profile of the endoscope shaft, a heating element disposed on the body and a power supply electrically coupled to a controller, wherein the controller operates the heating element.

In one aspect, the body may have a transverse axis with a radius of curvature to conform to the outer profile of the endoscope shaft. At least a portion of the body may define an interior diameter of greater than 180°. The endoscope heater may also have a retaining element configured to be attached to the endoscope shaft. Alternatively, at least a portion of the body may form a lumen through which the endoscope shaft may be advanced.

In one aspect, the body may be mounted to a substrate and the substrate may have a transverse axis with a radius of curvature to conform to the outer profile of the endoscope shaft. At least a portion of the substrate may define an interior diameter of greater than 180°. The endoscope heater may also have a retaining element configured to be attached to the endoscope shaft. Alternatively, at least a portion of the substrate may form a lumen through which the endoscope shaft may be advanced.

In one aspect, the endoscope heater is configured to be releasably attached to the endoscope.

In one aspect, the controller may be configured to maintain the heating element at a predetermined temperature.

In one aspect, the endoscope heater may also have a temperature sensor. Alternatively or in addition, the heating element may function as a temperature sensor.

In one aspect, the heating element may have a pattern formed from at least two materials.

This disclosure is also directed to a method for performing a procedure with an endoscope. The method may include providing an endoscope having an attached endoscope heater with an elongated body secured to a shaft of the endoscope, wherein a surface of the body conforms to an outer profile of the endoscope shaft, a heating element disposed on the body and a power supply electrically coupled to a controller, operating the controller to energize the heating element to warm the endoscope above ambient temperature and introducing the endoscope with the endoscope heater into a patient's body.

In one aspect, the controller may be operated to energize the heating element after introduction of the endoscope with the endoscope heater into the patient's body to avoid from fogging.

In one aspect, a temperature of the endoscope may be sensed, such that the controller receives feedback regarding the sensed temperature and selectively energizes the heating element to maintain a predetermined temperature.

In one aspect, the endoscope heater may be secured to the endoscope prior to operating the controller to energize the heating element to warm the endoscope above ambient temperature.

In one aspect, the endoscope heater may be detached from a first endoscope and secured to a second endoscope.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 depicts a schematic view of an embodiment of an endoscope heater on an endoscope.

FIG. 2 schematically depicts a partial sectional view of an embodiment of a heating element on a body.

FIG. 3 schematically depicts a view of an embodiment of a heating element having a specific pattern.

FIG. 4 schematically depicts a section view of the endoscope heater on the endoscope of FIG. 1.

FIG. 5 depicts a schematic view of an embodiment of an endoscope heater with a retaining element.

FIG. 6 schematically depicts a sectional view of another embodiment of an endoscope heater having a tubular body.

FIG. 7 depicts a schematic view of another embodiment of an endoscope heater on an endoscope in which the body is mounted to a substrate.

FIGS. 8-9 schematically depict sectional views of alternate embodiments of the endoscope heater of FIG. 7.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Notably, aspects of this disclosure are described in the context of an endoscope used to perform a laparoscopic procedure. However, different terms may be employed for procedures depending on the location of interest. As illustrations, endoscopy may refer to visualization of the digestive tract, colonoscopy may refer to visualization of the colon, arthroscopy may refer to visualization of a joint, laparoscopy may refer to visualization of the anatomy within the abdomen, thoracoscopy may refer to visualization of the anatomy within the chest, urethroscopy may refer to visualization of the urinary tract, bronchoscopy may refer to visualization of the respiratory tract, and other terms may be used depending on where the procedure is performed. Similarly, the instrument used for visualization may also be named in accordance with the location being viewed, using terms such as a gastroscope, pharyngoscope, laryngoscope, laparoscope, colonoscope. It should be appreciated that the techniques of this disclosure may be applied in conjunction with any instrument having an optical lens used to visualize the interior of a patient's body. Therefore, as used herein, the term “endoscope” is meant to include any viewing device or medical telescope that is inserted into the body of a subject and used to view internal structures.

Finally, as used in this specification and the appended claims, the singular forms “a, “an” and “the” include plural referents unless the content clearly dictates otherwise.

Referring to FIG. 1, an endoscope heater 1 according to one embodiment is shown. The endoscope heater 1 may be attached or secured to an endoscope 2 as an accessory without interfering with the primary function of the instrument, and in this embodiment, may be detachable or releasable from the endoscope 2, allowing for use with a different endoscope. In some applications, the entire endoscope heater 1 or some portion may be designed to be disposable. The endoscope heater 1 of this embodiment includes an elongated body 10, a heating element 11, a controller 12, and a power supply 13. The endoscope 2 has a shaft 21 with a distal end 211 and a proximal end 212 opposing each other. An objective lens 17 or an equivalent optical apparatus may be positioned at the distal end 211, while the proximal end 212 may have an eyepiece 18. The body 10 is configured to conform closely to the shaft of the endoscope 2 when in use. The size and shape of the body 10 may be adapted as appropriate to match the endoscope 2. For example, the endoscope 2 may be rigid or flexible. Rigid endoscopes 2, such as standard laparoscopes, usually have a shaft 21 of approximately 200-600 mm length, with an outer diameter of 3 mm to 25 mm. In other applications, the endoscope 2 may be over a meter in length and may permit flexion and manipulation of the distal end by the operator. The endoscope 2 may have any suitable objective lens orientation (such as 0°, 15°, 30°, 45°, 60°, and 70° endoscopes). The shaft 21 of the endo scope 2 often contains light-transmitting fiber-optic bundles and/or lenses that transmit visual signals and light. Accordingly, the body 10 may be of sufficient length to extend along the shaft 21. In the present embodiment, the body 10 can be made of insulated material, such as polyimide (PI), polyester (PET), polyvinyl chloride (PVC), silicon, mica or any combination thereof. The body 10 may also be formed from suitable metals, including stainless steel, or shape memory materials such as nickel-titanium alloys.

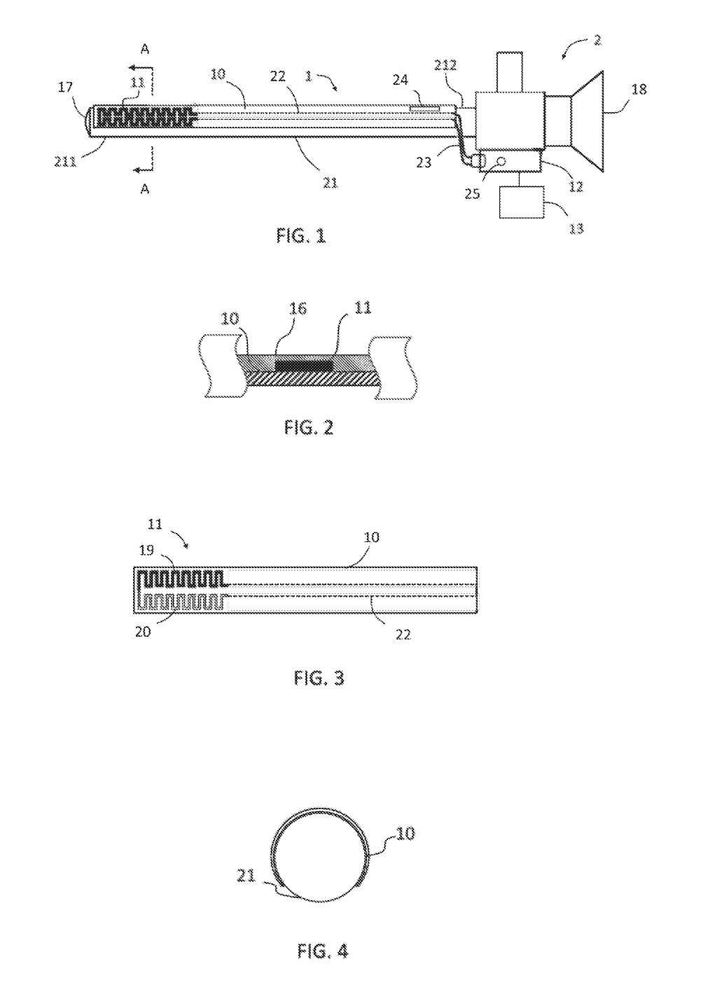

A partial cross sectional view of the heating element 1 without the endoscope 2, taken along line A-A in FIG. 1, is shown in FIG. 2. In one embodiment, the heating element 11 may be disposed on the body 10 opposing the surface that abuts the shaft 21 of the endoscope 2. Conversely, the heating element 11 may be disposed on the same surace that abuts the shaft 21. In addition, the heating element 11 on the body 10 can further be covered by a layer of insulation 16 for protection as shown. The layer of insulation 16 is optional and may be omitted if desired. The material of insulation layer 16 may be chosen from a material with good thermal conductive properties, e.g. thermal conductive adhesive. In this case, the insulation layer 16 can be applied to adhere the body 10 with the heating element 11 to the shaft 21 of endoscope 2. The heating element 11 is configured to function as a heat source and may be made of electrical conductive gel, electric wire, oxide semiconductor, metal alloy foil, carbon nanotube or any combination thereof. The heating element 11 can be manufactured by etching or printing to have a specific pattern as warranted. In the depicted embodiment, the heating element 11 is made of metal alloy foil.

In another embodiment, as shown in FIG. 3, the heating element 11 may comprise a pattern of at least two materials 19 and 20. In an embodiment, the material 19 can be nichrome and the material 20 can be copper. The heating element 11 may be coupled to the controller 12 through conductors 22 (shown in phantom) embedded within the body 10.

As shown, the heating element 11 may be formed at one end of the body 10. Correspondingly, the heating element 11 may be positioned so that it is adjacent the distal end 211 of the endoscope 2. Thus, the heat generated by the heating element 11 may more readily increase the temperature around the distal end 211 of the endoscope 2, including the lens 17. Alternatively, the heating element 11 may be deployed longitudinally along the body 10 and be configured to distribute heat substantially evenly along the shaft 21.

Referring back to FIG. 1, leads 23 may connect the controller 12 to the heating element 11 through conductors 22 (also shown here in phantom) as noted. The controller 12 may be connected to the power supply 13 and include a heating circuit to control a temperature of the heating element 11. Any suitable electrical connection may be made between the heating element 11 and the controller 12 to allow operation by the heating circuit. In some embodiments, leads 23 of the controller 12 may use a detachable connector to couple with the body 10, so that the body 10 with heating element 11 may easily plug into the controller 12. By employing this configuration, the controller 12 and the power supply 13 may be reused, while the body 10 may be disposable as noted above.

The heating circuit of the controller 12 may be designed to control the heating element 11 to ramp up to a desired temperature according to the intended use. For example, the heating circuit controls the heating element 11 to approach and then maintain a predetermined temperature ranging from about 33° C. to 41° C.

The endoscope heater 1 may also include a temperature sensor 24 electrically connected to the controller 12 by conductors 22. The sensor 24 can be placed in a variety places of the endoscope heater 1 to coordinate with predetermined thermal control. As shown in FIG. 1, the sensor 24 may be disposed on the body 10 adjacent to the proximal end 212 of shaft 21. This design may minimize the diameter of the endoscope heater 1 at its distal end. However, in other embodiments, the sensor 24 may be placed adjacent to the lens 17 to more accurately detect the temperature of the lens 17. The signal generated from the sensor 24 is transmitted to the controller 12, allowing for feedback control of the heating element 11. Alternatively, the heating element 11 itself may be designed as a temperature sensor using the intrinsic property of the heating element 11 whose resistance is dependence on the temperature. In such embodiments, a dedicated sensor is not required, allowing for a reduced overall diameter and a less complex structure.

The power supply 13 may be electrically connected to the heating element 11 as noted above and may be a battery or a AC converter as desired. In addition, the endoscope heater 1 may also include a switch 25 electrically coupled to the heating circuit to actuate the heating element 11.

In use, the endoscope heater 1 may be attached to the endoscope 2. The operator powers on the heating element 11 to ramp up to a predetermined temperature in a range of about 33° C. to 41° C., before surgery. The controller 12 may power off heating the heating element 11 when the temperature is above the predetermined temperature, e.g. 41° C., to maintain the lens 17 at the desired temperature. During surgery, the sensor 24 may detect a change of temperature and transmit the signal to the controller 12. The controller 12 then reenergizes the heating element 11 to provide heat when the temperature falls below the predetermined temperature. Alternatively, the sensor 24 may be omitted to save expense and the controller 12 simplified to energize the heating element 11 continuously. By employing an endoscope heater 1 having the features of this disclosure, an endoscope, such as the endoscope 2, may be warmed above the temperature of the ambient environment so that when introduced into the patient, the temperature differential between the endoscope 2 and the patient's body cavity may be minimized, avoiding the tendency of lens 17 fogging. Moreover, the ant-fogging effect may be maintained through use of the controller 12 to monitor and keep the temperature of the endoscope 2 in a desired temperature range as described above.

It is generally desirable to have the body 10 attach securely to the endoscope 2 so that it is not dislodged during introduction into the patient's body as well as to improve the transfer of heat from the heating element 11. As shown in FIG. 1, the body 10 may extend from the distal end 211 of the shaft 21 to the proximal end 212, covering at least a portion of the shaft 21. The body 10 may have a shape that conforms to the profile of the shaft 21. For example, the shaft 21 may have a generally circular outer profile and the body 10 may have a correspondingly curved or arc shape, such that its transverse axis has a suitable radius of curvature that matches the profile of the shaft 21. Referring to FIG. 4, which is a complete cross sectional view taken along line A-A of FIG. 1, the body 10 may extend around at least a portion of the diameter of the shaft 21. In some embodiments, the body 10 may extend around more than half the diameter so as to retain the shaft 21. For example, the body 10 may define an interior diameter of greater than about 180°. When the body 10 is formed from a suitably resilient material, it may be snap fit over the shaft 21. Alternatively, the shaft 21 may be advanced coaxially within the body 10 when the endoscope heater 1 is installed. Accordingly, the body 10 may be in intimate contact with the surface of the shaft 21 and conform closely to it, providing good thermal contact.

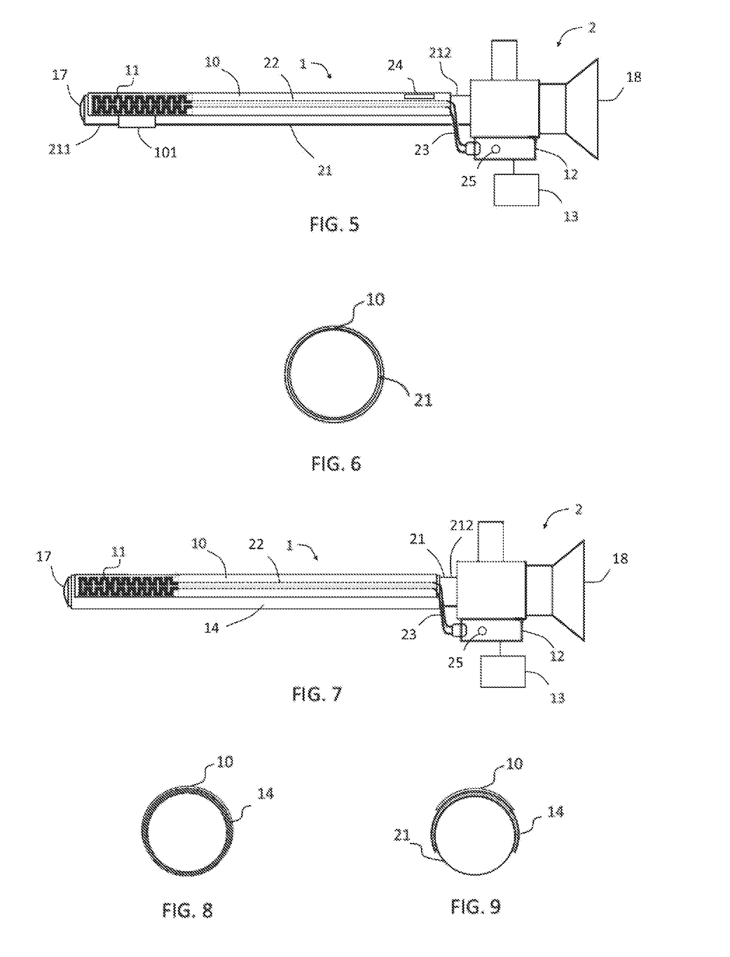

In the embodiment shown in FIG. 5, the endoscope heater 1 may also include a retaining element 101 to provide an additional degree of attachment to the endoscope 2. As shown, retaining element 101 may cooperate with the body 10 to form a ring that encircles the shaft 21. The retaining element 101 may be a radial extension of the body 10 or may be formed as a separate structure that is then suitably secured to the body 10. In use, the operator may pass the shaft 21 of the endoscope 2 through the ring formed by the retaining element 101 and the body 10. The retaining element 101 provides an enhanced engagement between the body 10 and the endoscope. Other configurations may also be employed for retaining element 101, such in the form of opposing projections that at least partially encircle the shaft 21. In one aspect, the combinations of projections and the body 10 encircle more than half the diameter of the shaft 21 to provide a retention force. As another illustration, the retaining element 101 may be a structure that mates with a cooperating structure on the shaft 21. In a further example, the retaining element 101 may comprise heat-shrink tubing that may be deployed over the body 10 and the shaft 21 and shrunk to secure them together.

In another aspect, the body 10 may have a tubular configuration as shown schematically in cross section in FIG. 6. The body 10 may define a lumen through which the shaft 21 may pass through, so that the body 10 retains and is secured to the shaft 21. For example, an inside diameter of the body 10 may be substantially equal to an outside diameter of the shaft 21 of the endoscope 2 to provide a reliable attachment. The body 10 may be a hollow cylindrical body structure having a wall thickness less than or equal to about 3 mm. The wall thickness of the body 10 according to another embodiment may be less than or equal to about 1 mm. Alternatively, the body 10 may have a wall thickness less than or equal to about 0.4 mm. Moreover, the body 10 of still another embodiment may have a wall thickness less than or equal to about 0.2 mm. In other applications, the wall thickness of the body 10 may be thinner as warranted.

To help illustrate another aspect of this disclosure, FIG. 7 depicts an embodiment in which the body 10 is disposed on a substrate 14, which is then attached to the shaft 21. The substrate 14 may be made of plastic or stainless steel, for example. Although shown as being mounted on an exterior surface of the substrate 14, the body 10 may also be mounted on an interior surface of the substrate 14, so that the body 10 is coaxially disposed between the shaft 21 and the substrate 14. The substrate 14 may also have a size and shape configured to facilitate the attachment, such as by employing the techniques described above with regard to the body 10. For example, the substrate 14 may have a tubular structure as shown in the schematic cross section of FIG. 8. The relative diameters of the elements may be adjusted to increase the degree of attachment. For example, the curved shape of the body 10 may provide a radial force to attache the heating element 11 to the substrate 14. The diameter of the substrate 14 may be reduced to help position the body 10 as desired. As such, the substrate 14 may be a hollow cylindrical body structure having a wall thickness less than or equal to about 3 mm. The wall thickness of the substrate 14 according to another embodiment may be less than or equal to about 1 mm. Alternatively, the substrate 14 may have a wall thickness less than or equal to about 0.4 mm. Moreover, the substrate 14 of still another embodiment may have a wall thickness less than or equal to about 0.2 mm. In other applications, the wall thickness of the substrate 14 may be thinner as warranted.

In another embodiment as shown in FIG. 9, the substrate 14 may only partially encircle the shaft 21, such as more than half the diameter. By closely fitting the radius of curvature of the substrate 14 to the outer diameter of the shaft 21, a suitable degree of retention may be achieved. To provide additional security, the substrate 14 may also have a retaining element, similar in function to the retaining element 101 described above with respect to the body 10.

Described herein are certain exemplary embodiments. However, one skilled in the art that pertains to the present embodiments will understand that the principles of this disclosure can be extended easily with appropriate modifications to other applications.

What is claimed is:

1. An endoscope heater comprising:
an elongated body configured to be attached to an endoscope shaft, a surface of the body conforming to an outer profile of the endoscope shaft such that a lens of the endoscope remains exposed when the elongated body is attached to the endoscope shaft;
a heating element disposed on the body; and
a power supply electrically coupled to a controller, wherein the controller operates the heating element.

2. The endoscope heater of claim 1, wherein the body has a transverse axis with a radius of curvature to conform to the outer profile of the endoscope shaft.

3. The endoscope heater of claim 2, wherein at least a portion of the body defines an interior diameter of greater than 180°.

4. The endoscope heater of claim 2, further comprising a retaining element configured to be attached to the endoscope shaft.

5. The endoscope heater of claim 2, wherein at least a portion of the body forms a lumen through which the endoscope shaft may be advanced.

6. The endoscope heater of claim 1, wherein the body has a wall thickness less than or equal to about 0.4 mm.

7. The endoscope heater of claim 1, wherein the body is made of insulated material.

8. The endoscope heater of claim 1, further comprising a layer of insulation covering the heating element.

9. The endoscope heater of claim 1, further comprising a substrate, wherein the body is mounted to the substrate and wherein the substrate has a transverse axis with a radius of curvature to conform to the outer profile of the endoscope shaft.

10. The endoscope heater of claim 9, wherein at least a portion of the substrate defines an interior diameter of greater than 180°.

11. The endoscope heater of claim 9, further comprising a retaining element configured to be attached to the endoscope shaft.

12. The endoscope heater of claim 9, wherein at least a portion of the substrate forms a lumen through which the endoscope shaft may be advanced.

13. The endoscope heater of claim 1, wherein the endoscope heater is configured to be releasably attached to the endoscope shaft.

14. The endoscope heater of claim 1, wherein the controller is configured to maintain the heating element at a predetermined temperature.

15. The endoscope heater of claim 14, further comprising a temperature sensor electrically connected to the controller.

16. The endoscope heater of claim 1, wherein the heating element is configured to function as a temperature sensor.

17. The endoscope heater of claim 1, wherein the heating element comprises a pattern formed from at least two materials.

18. A method for performing a procedure with an endoscope, comprising:
providing an endoscope having: i) an attached endoscope heater with an elongated body secured to a shaft of the endoscope, a surface of the elongated body conforming to an outer profile of the endoscope shaft such that a lens of the endoscope remains exposed when the elongated body is attached to the endoscope shaft; ii) a heating element disposed on the body; and iii) a power supply electrically coupled to a controller;
operating the controller to energize the heating element to warm the endoscope above ambient temperature; and
introducing the endoscope with the endoscope heater into a patient's body.

19. The method of claim 18, further comprising operating the controller to energize the heating element after introduction of the endoscope into the patient's body to avoid from fogging of the lens.

20. The method of claim 18, further comprising sensing a temperature of the endoscope, wherein the controller receives feedback regarding the sensed temperature and selectively energizes the heating element to maintain a predetermined temperature.

21. The method of claim 18, further comprising securing the endoscope heater to the endoscope prior to operating the controller to energize the heating element to warm the endoscope above ambient temperature.

22. The method of claim 18, further comprising detaching the endoscope heater from a first endoscope and securing the endoscope heater to a second endoscope.
